(19) United States
(12) Patent Application Publication
(10) Pub. No.: US 2013/0131090 A1
(43) Pub. Date: May 23, 2013

(54) SALTS OF LAPATINIB

(71) Applicants: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Thungathurthy Srinivasa Rao, Hyderabad (IN); Bandi Vamsi Krishna, Hyderabad (IN)

(72) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Thungathurthy Srinivasa Rao, Hyderabad (IN); Bandi Vamsi Krishna, Hyderabad (IN)

(21) Appl. No.: 13/813,318

(22) PCT Filed: Aug. 1, 2011
(86) PCT No.: PCT/IN11/00505

§ 371 Date: Jan. 30, 2013

(30) Foreign Application Priority Data
Aug. 3, 2010 (IN) 2201/CHE/2010

(51) Int. Cl. C07D 405/10

(52) U.S. Cl. CPC C07D 405/10
USPC 514/266.24; 544/287

(57) ABSTRACT

The present invention provides novel dioxalate salt of lapatinib, process for its preparation and pharmaceutical compositions comprising it. The present invention also provides novel monobesylate salt of lapatinib, process for its preparation and pharmaceutical compositions comprising it. The present invention further provides a process for the preparation of monohydrate form of lapatinib ditosylate. The present invention further provides a process for the preparation of anhydrous form of lapatinib ditosylate.

FIELD OF THE INVENTION The present invention provides novel dioxalate salt of lapatinib, process for its preparation and pharmaceutical compositions comprising it. The present invention also provides novel monobesylate salt of lapatinib, process for its preparation and pharmaceutical compositions comprising it. The present invention further provides a process for the preparation of monohydrate form of lapatinib ditosylate. The present invention further provides a process for the preparation of anhydrous form of lapatinib ditosylate.

### BACKGROUND OF THE INVENTION

Lapatinib is chemically, N-[3-chloro-4[(3-fluorophenyl)methoxy]phenyl]-6-[5-[(2-methylsulfonylethylamino)methyl]-2-furyl]quinazolin-4-amine and has the structural formula:

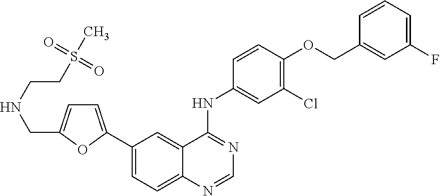 [SMILES: CS(=O)(=O)CCNCc1ccc(-c2ccc3ncnc(Nc4ccc(OCc5cccc(F)c5)c(Cl)c4)c3c2)o1]

Lapatinib ditosylate is currently marketed in the United States under the tradename TYKERB® by GlaxoSmithKline.

Polymorphism is defined as “the ability of a substance to exist as two or more crystalline phases that have different arrangement and/or conformations of the molecules in the crystal Lattice. Thus, in the strict sense, polymorphs are different crystalline forms of the same pure substance in which the molecules have different arrangements and/or different configurations of the molecules”. Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning Calorimetry (DSC) and Infrared spectrometry (IR).

Solvent medium and mode of crystallization play very important role in obtaining a crystalline form over the other.

Lapatinib and its salts can exist in different polymorphic forms, which may differ from each other in terms of stability, physical properties, spectral data and methods of preparation.

Lapatinib ditosylate was described in PCT publications WO 1999/035146, WO 2002/002552, WO 2005/046678, WO 2006/113649, WO 1998/002437, WO 2001/004111, WO 1996/009294, WO 2002/056912, WO 2005/105094, WO 2005/120504, WO 2005/120512, WO 2006/026313 and WO 2006/066267.

U.S. Pat. No. 7,157,466 disclosed anhydrous and monohydrate forms of lapatinib ditosylate.

PCT Publication WO 2008/154469 disclosed di and mono esylate, di and mono mesylate, di and mono L-lactate, di and mono L-malate, dimaleate, bibenzoate, di and mono L-tartrate, monocitrate, fumarate, dibenzoate, di and mono L-tartrate, monocitrate, fumarate, dibesylate, hydrobromide, salicylate, succinate and diesylate salts of lapatinib.

Lapatinib hydrochloride salt was disclosed in U.S. Pat. No. 6,727,256.

PCT publication WO 2009/137714 disclosed crystalline form I, form II, form III, form IV, form V, form VI, form VII, form VIII, form IX, form XI, form XII, form XIII, form XIV, form XV, form XVI, form XVII, form XVIII and form XIX of lapatinib ditosylate.

U.S. patent application no. 2010/0087459 disclosed monotosylate, sulfate, dihydrobromide and phosphate salts of lapatinib.

We have discovered novel salts of lapatinib such as dioxalate and monobesylate, and also discovered a process for the preparation of monohydrate form of lapatinib ditosylate and a process for the preparation of anhydrous form of lapatinib ditosylate.

Thus, one object of the present invention is to provide a dioxalate salt of lapatinib, process for its preparation and pharmaceutical composition comprising it.

Another object of the present invention is to provide a monobesylate salt of lapatinib, process for its preparation and pharmaceutical composition comprising it.

The salt of the present invention may also serve as intermediate for preparation of lapatinib free base or another salt of lapatinib.

Another object of the present invention is to provide a process for the preparation of monohydrate form of lapatinib ditosylate.

Yet another object of the present invention is to provide a process for the preparation of anhydrous form of lapatinib ditosylate.

### SUMMARY OF THE INVENTION

In one aspect, the present invention provides a dioxalate salt of lapatinib, that is, lapatinib dioxalate.

In another aspect, the present invention provides a process for the preparation of lapatinib dioxalate, which comprises:
a) dissolving lapatinib in an ether solvent;
b) heating the contents to an elevated temperature;
c) adding oxalic acid to the solution obtained in step (b); and
d) isolating lapatinib dioxalate.

In another aspect, the present invention provides a pharmaceutical composition comprising lapatinib dioxalate and a pharmaceutically acceptable excipient.

In another aspect, the present invention provides a monobesylate salt of lapatinib, that is, lapatinib monobesylate.

In another aspect, the present invention provides a process for the preparation of lapatinib monobesylate, which comprises:
a) dissolving lapatinib in a nitrile solvent;
b) heating the contents to an elevated temperature;
c) adding benzenesulfonic acid to the solution obtained in step (b); and
d) isolating lapatinib monobesylate.

In another aspect, the present invention provides a pharmaceutical composition comprising lapatinib monobesylate and a pharmaceutically acceptable excipient.

In another aspect, the present invention provides a process for the preparation of monohydrate form of lapatinib ditosylate, which comprises:
a) dissolving lapatinib in a ketonic solvent and optionally adding water;
b) heating the contents to an elevated temperature;
c) adding p-toluenesulfonic acid to the solution obtained in step (b);
d) slurrying the reaction mass obtained in step (c) at about 25 to 35° C.; and
e) isolating monohydrate form of lapatinib ditosylate.

Yet another aspect, the present invention provides a process for the preparation of anhydrous form of lapatinib ditosylate, which comprises:
a) suspending lapatinib in an ester solvent;
b) heating the contents to an elevated temperature;
c) adding p-toluenesulfonic acid to the solution obtained in step (b); and
d) isolating anhydrous form of lapatinib ditosylate.

### BRIEF DESCRIPTION OF THE DRAWING

FIG. 1 is X-ray powder diffraction spectrum of amorphous lapatinib dioxalate.

FIG. 2 is X-ray powder diffraction spectrum of crystalline lapatinib monobesylate.

FIG. 3 is X-ray powder diffraction spectrum of monohydrate form of lapatinib ditosylate.

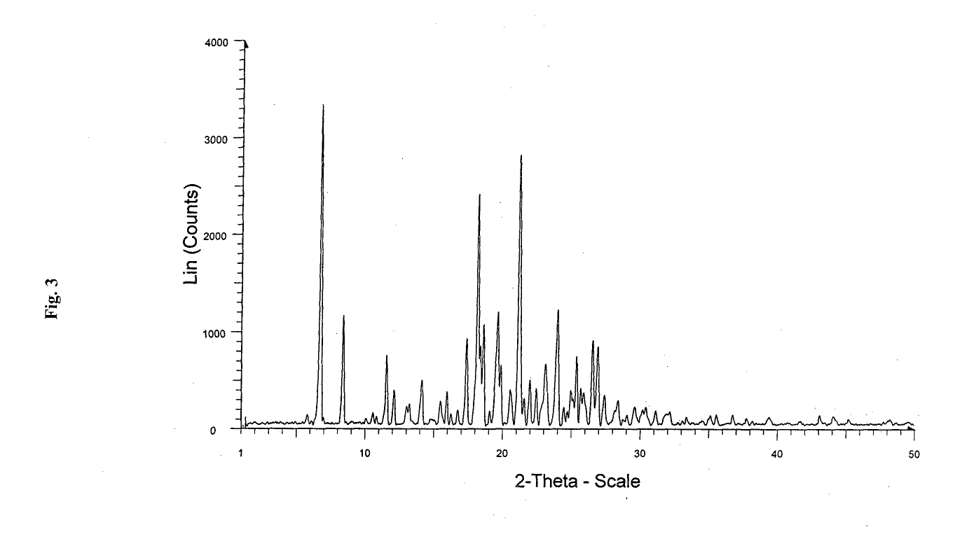

FIG. 4 is X-ray powder diffraction spectrum of anhydrous form of lapatinib ditosylate.

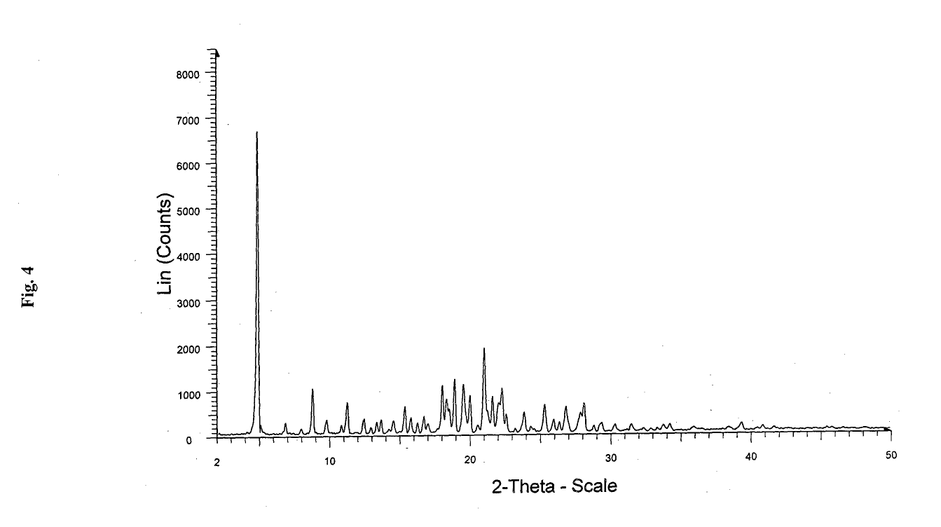

X-ray powder diffraction spectrum was measured on a bruker axs D8 advance X-ray powder diffractometer having a copper-Kα radiation. Approximately 1 gm of sample was gently flattered on a sample holder and scanned from 2 to 50 degrees two-theta, at 0.02 degrees to theta per step and a step of 52 seconds. The sample was simply placed on the sample holder. The sample was rotated at 30 rpm at a voltage 40 KV and current 35 mA.

### DETAILED DESCRIPTION OF THE INVENTION

According to one aspect of the present invention, there is provided a dioxalate salt of lapatinib, that is, lapatinib dioxalate.

The lapatinib dioxalate may preferably be a solid.

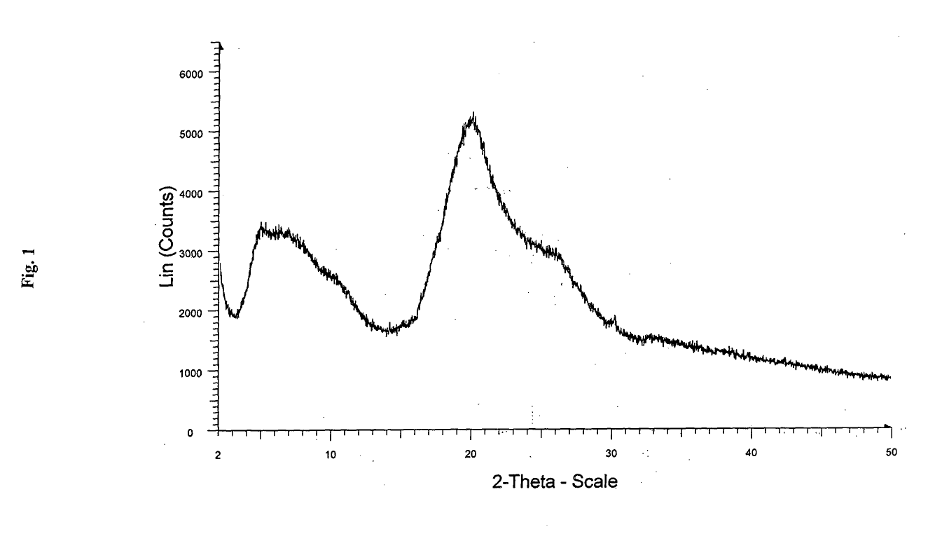

The powdered x-ray diffractogram (PXRD) of amorphous lapatinib dioxalate is shown in FIG. 1.

According to another aspect of the present invention, there is provided a process for the preparation of lapatinib dioxalate, which comprises:
a) dissolving lapatinib in an ether solvent;
b) heating the contents to an elevated temperature;
c) adding oxalic acid to the solution obtained in step (b); and
d) isolating lapatinib dioxalate.

The ether solvent used in the process may preferably be a solvent or mixture of solvents selected from tetrahydrofuran, 1,4-dioxane, tert-butyl methyl ether and diethyl ether. More preferable ether solvent is tetrahydrofuran.

The term “elevated temperature” refers to temperature at above 25° C. Preferably the contents are heating in step (b) at about 60 to 70° C.

Lapatinib dioxalate may be isolated in step (d) by methods known such as filtration or centrifugation.

According to another aspect of the present invention, there is provided a pharmaceutical composition that comprises lapatinib dioxalate and pharmaceutically acceptable carriers, diluents or excipients and optionally other therapeutic ingredients. The salt may preferable be conveniently formulated into tablets, capsules, suspensions, dispersions, injectables and other pharmaceutical forms.

According to another aspect of the present invention, there is provided a monobesylate salt of lapatinib, that is, lapatinib monobesylate.

The lapatinib monobesylate may preferably be a solid and more preferable solid is crystalline lapatinib monobesylate.

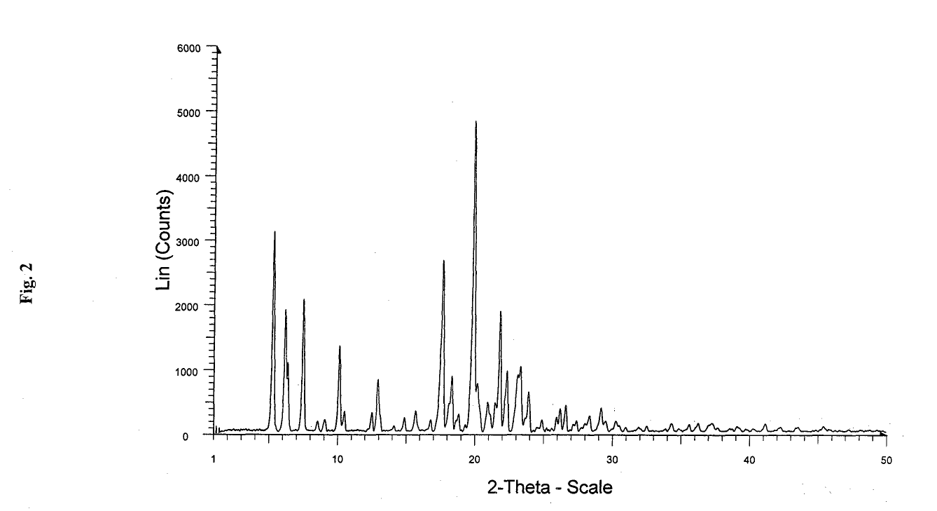

The powdered x-ray diffractogram (PXRD) of crystalline lapatinib monobesylate is shown in FIG. 2.

According to another aspect of the present invention, there is provided a process for the preparation of lapatinib monobesylate, which comprises:
a) dissolving lapatinib in a nitrile solvent;
b) heating the contents to an elevated temperature;
c) adding benzenesulfonic acid to the solution obtained in step (b); and
d) isolating lapatinib monobesylate.

The nitrile solvent used in the process may preferably be a solvent or mixture of solvents selected from acetonitrile, propionitrile, butyronitrile and benzonitrile. More preferable nitrile solvent is acetonitrile.

The tenn “elevated temperature” refers to temperature at above 25° C. Preferably the contents are heating in step (b) at about 60 to 70° C.

Lapatinib monobesylate may be isolated in step (d) by methods known such as filtration or centrifugation.

According to another aspect of the present invention, there is provided a pharmaceutical composition that comprises lapatinib monobesylate and pharmaceutically acceptable carriers, diluents or excipients and optionally other therapeutic ingredients. The salt may preferable be conveniently formulated into tablets, capsules, suspensions, dispersions, injectables and other pharmaceutical forms.

According to another aspect of the present invention, there is provided a process for the preparation of monohydrate form of lapatinib ditosylate, which comprises:
a) dissolving lapatinib in a ketonic solvent and optionally adding water;
b) heating the contents to an elevated temperature;
c) adding p-toluenesulfonic acid to the solution obtained in step (b);
d) slurrying the reaction mass obtained in step (c) at about 25 to 35° C.; and
e) isolating monohydrate form of lapatinib ditosylate.

The ketonic solvent used in step (a) may preferably be a solvent or mixture of solvents selected from acetone, methyl ethyl ketone, methyl isobutyl ketone and diethyl ketone. More preferable ester solvent is acetone.

The term “elevated temperature” refers to temperature at above 25° C. Preferably the contents are heating in step (b) at reflux.

Isolation of monohydrate form of lapatinib ditosylate in step (e) may preferably be performed by conventional techniques such as centrifugation and filtration.

According to another aspect of the present invention, there is provided a process for the preparation of anhydrous form of lapatinib ditosylate, which comprises:
a) suspending lapatinib in an ester solvent;
b) heating the contents to an elevated temperature;
c) adding p-toluenesulfonic acid to the solution obtained in step (b); and
d) isolating anhydrous form of lapatinib ditosylate.

The ester solvent used in step (a) may preferably be a solvent or mixture of solvents selected from ethyl acetate, methyl acetate, isopropyl acetate, tert-butyl methyl acetate and ethyl formate. More preferable ester solvent is isopropyl acetate.

The term “elevated temperature” refers to temperature at above 25° C. Preferably the contents are heating in step (b) at reflux.

Isolation of anhydrous form of lapatinib ditosylate in step (d) may preferably be performed by conventional techniques such as centrifugation and filtration.

The invention will now be further described by the following examples, which are illustrative rather than limiting.

### EXAMPLES

### Example 1

### Preparation of Lapatinib

2-Fluraldehyde diethyl acetal (40 gm) was dissolved in dimethoxy ethane (270 ml) at room temperature under nitrogen atmosphere and then cooled to −40° C. N-Butyl lithium (180 ml) was added to the solution for 45 minutes and stirred for 2 hours at −40 to −35° C. To the reaction mass was added triisopropyl borate (53 gm) for 30 minutes and stirred for 2 hours at −40 to −35° C. The temperature of the reaction mass was raised to 0° C. and then added acetic acid (12 ml), stirred for 30 minutes at 0° C. To the reaction mass was added water (15 ml) and stirred for 15 minutes. A mixture of ethanol (200 ml), triethylamine (41 ml) and N-{3-chloro-4-[(3-fluorobenzyl)oxy}phenyl}-6-iodo-4-quinazolinamine (59 gm) was added to the above reaction mass at 20 to 25° C. and then added palladium carbon (5%, 3.5 gm). The contents were heated to 60 to 65° C. and maintained for 4 hours 60 to 65° C. The reaction mass was cooled to room temperature and maintained for 30 minutes at room temperature. The reaction mass was filtered through hi-flo bed and the filtrate was cooled to 20 to 25° C. To the reaction mass was added p-toluenesulfonic acid (91 gm) and stirred for 1 hour at room temperature. The separated solid was filtered and dried under vacuum at 50 to 55° C. for 5 hours to obtain 60 gm of 5-[4-({3-chloro-4-{(3-fluoropheny)methoxy]phenyl)amino)quinazolin-6-yl]furan-2-carbaldehyde p-toluenesulfonic acid.

5-[4-({3-Chloro-4-{(3-fluorophenyl)methoxy]phenyl)amino)quinazolin-6-yl]furan-2-carbaldehyde p-toluenesulfonic acid as obtained above, tetrhydrofuran (1000 ml), 2-(methanesulphonyl)ethylamine (40 gm) and acetic acid (35 ml) were added at room temperature. Diisopropylethylamine (108 ml) was added to the reaction mass and stirred for 2 hours at 30 to 35° C., and then cooled to 20° C. To the reaction mass was added sodium triacetoxy borohydride (66 gm) and maintained for 3 hours at 20 to 25° C., and then added a mixture of sodium hydroxide solution (25%, 310 ml) and water (200 ml). The layers were separated and aqueous layer was extracted with tetrahydrofuran. The combined organic layer was dried over sodium sulfate and the solvent was distilled off under vacuum at below 50° C. to obtain residual mass. To the residual mass was added isopropyl acetate (300 ml) and stirred for 30 minutes at 55 to 60° C. The reaction mass was cooled to room temperature and stirred for 30 minutes at room temperature, filtered. The solid obtained was dried under vacuum at 50 to 55° C. for 6 hours to obtain 78 gm of crude lapatinib.

Crude lapatinib as obtained above was dissolved in methanol (390 ml) and dichloromethane (780 ml) and then treated with carbon (7 gm) at room temperature. The reaction mass was stirred for 20 minutes and filtered through hi-flo bed. The solvent was distilled off under vacuum at 45 to 50° C. to obtain residual mass. To the residual mass was added methanol (50 ml) and stirred for 1 hour at room temperature. The separated solid was filtered and dried under vacuum at 50 to 55° C. for 6 hours to obtain 66 gm of lapatinib.

### Example 2

### Preparation of Lapatinib Dioxalate

Lapatinib (4 gm) as obtained in example 1 was dissolved in tetrahydrofuran (40 ml) at 25 to 30° C. The contents were heated to 65 to 70° C. to obtain a solution. To the solution was added oxalic acid (1.6 gm) and stirred for 1 hour at 65 to 70° C. The reaction mass was cooled to 25 to 30° C. and stirred for 1 hour at 25 to 30° C. The solid obtained was collected by filtration and dried under vacuum at 55 to 60° C. for 6 hours to obtain 4.2 gm of lapatinib dioxalate.

### Example 3

### Preparation of Lapatinib Dioxalate

Lapatinib (48 gm) was dissolved in tetrahydrofuran (480 ml) at 25 to 30° C. and then heated to 65 to 70° C. to obtain a solution. To the solution was added oxalic acid (19 gm) and stirred for 1 hour at 65 to 70° C. The reaction mass was cooled to 25 to 30° C. and stirred for 1 hour at 25 to 30° C., filtered. The solid obtained was dried under vacuum at 55 to 60° C. for 6 hours to obtain 47 gm of lapatinib dioxalate.

### Example 4

### Preparation of Lapatinib Dioxalate

Lapatinib (4 gm) was dissolved in tetrahydrofuran (40 ml) at 25 to 30° C. and then heated to 65 to 70° C. to obtain a solution. To the solution was added oxalic acid (1.3 gm) and stirred for 1 hour at 65 to 70° C. The reaction mass was cooled to 25 to 30° C. and stirred for 1 hour at 25 to 30° C. The solid obtained was collected by filtration and dried under vacuum at 55 to 60° C. for 6 hours to obtain 4 gm of lapatinib dioxalate.

### Example 5

### Preparation of Lapatinib Monobesylate

Lapatinib (5 gm) was dissolved in acetonitrile (200 ml) at 25 to 30° C. and then heated to 65 to 70° C. to obtain a solution. To the solution was added benzene sulphonic acid (1.6 gm) and stirred for 1 hour at 65 to 70° C. The reaction mass was cooled to 25 to 30° C. and stirred for 1 hour at 25 to 30° C. The solid obtained was collected by filtration and dried under vacuum at 55 to 60° C. for 7 hours to obtain 6 gm of lapatinib monobesylate.

### Example 6

### Preparation of Lapatinib Monobesylate

Lapatinib (50 gm) was dissolved in acetonitrile (2000 ml) at 25 to 30° C. and then heated to 65 to 70° C. to obtain a solution. To the solution was added benzene sulphonic acid (16 gm) and stirred for 1 hour at 65 to 70° C. The reaction mass was cooled to 25 to 30° C. and stirred for 1 hour at 25 to 30° C., filtered. The solid obtained was dried under vacuum at 55 to 60° C. for 7 hours to obtain 59 gm of lapatinib monobesylate.

### Example 7

### Preparation of Lapatinib Monobesylate

Lapatinib (5 gm) was dissolved in acetonitrile (200 ml) at 25 to 30° C. The contents were heated to 65 to 70° C. to obtain a solution. To the solution was added benzene sulphonic acid (1.4 gm) and stirred for 1 hour at 65 to 70° C. The reaction mass was cooled to 25 to 30° C. and stirred for 1 hour at 25 to 30° C. The solid obtained was collected by filtration and dried under vacuum at 55 to 60° C. for 7 hours to obtain 5.8 gm of lapatinib monobesylate.

### Example 8

### Preparation of Monohydrate Form of Lapatinib Ditosylate

Lapatinib (4 gm) was dissolved in acetone (60 ml) water (4 ml) at 25 to 30° C. The contents were heated to reflux and then added p-toluenesulfonic acid (2.8 gm), stirred for 1 hour at 65 to 70° C. The reaction mass was cooled to 25 to 30° C. and stirred for 16 hour at 25 to 30° C. The separated solid was filtered and dried under vacuum at 55 to 60° C. for 7 hours to obtain 5.2 gm of monohydrate form of lapatinib ditosylate.

### Example 9

### Preparation of Anhydrous Form of Lapatinib Ditosylate

Lapatinib (63 gm) was dissolved in isopropyl acetate (1575 ml) at 25 to 30° C. The contents were heated to reflux and then added p-toluenesulfonic acid (44 gm). The reaction mass was stirred for 1 hour at 65 to 70° C. and then cooled to 25 to 30° C. The reaction mass was stirred for 1 hour at 25 to 30° C. and filtered. The solid obtained was dried under vacuum at 50 to 55° C. for 7 hours to obtain 75 gm of anhydrous form of lapatinib ditosylate.

1. A dioxalate salt of lapatinib or monobesylate salt of lapatinib.

2. A process for the preparation of lapatinib dioxalate, comprising:
a. suspending lapatinib in an ether solvent to form a suspension;
b. heating the suspension to an elevated temperature to form a solution;
c. adding oxalic acid to the solution obtained in step (b); and
d. isolating lapatinib dioxalate from the solution of step (c).

3. The process according to claim 2, wherein the ether solvent used in the process is selected from the group consisting of tetrahydrofuran, 1,4-dioxane, tert-butyl methyl ether, diethyl ether, and mixtures thereof.

4. The process according to claim 3, wherein the ether solvent is tetrahydrofuran.

5. The process according to claim 2, wherein heating in step (b) is above 25° C.

6. The process according to claim 5, wherein heating is at 60 to 70° C.

7. (canceled)

8. A process for the preparation of lapatinib monobesylate, comprising:
a. dissolving lapatinib in a nitrile solvent to form a solution;
b. heating the solution to an elevated temperature;
c. adding benzenesulfonic acid to the solution obtained in step (b); and
d. isolating lapatinib monobesylate from the solution from the solution of step (c).

9. The process according to claim 8, wherein the nitrile solvent used in the process is selected from the group consisting of acetonitrile, propionitrile, butyronitrile, benzonitrile, and mixtures thereof.

10. The process according to claim 9, wherein the nitrile solvent is acetonitrile.

11. The process according to claim 8, wherein heating in step (b) is at above 25° C.

12. The process according to claim 11, wherein heating is at 60 to 70° C.

13. A process for the preparation of monohydrate form of lapatinib ditosylate, comprising:
a. suspending lapatinib in a ketonic solvent and optionally adding water to form a suspension;
b. heating the suspension to an elevated temperature to form a solution ;
c. adding p-toluenesulfonic acid to the solution obtained in step (b) to form a reaction mass;
d. slurrying the reaction mass obtained in step (c) at below 40° C.; and
e. isolating the monohydrate form of lapatinib ditosylate from the slurry.

14. The process according to claim 13, wherein the ketonic solvent is selected from the group consisting of acetone, methyl ethyl ketone, methyl isobutyl ketone, diethyl ketone, and mixtures thereof.

15. (canceled)

16. The process according to claim 13, wherein heating in step (b) is at above 25° C.

17. The process according to claim 16, wherein heating in step (b) is at reflux.

18. A process for the preparation of anhydrous form of lapatinib ditosylate, comprising:
a. suspending lapatinib in an ester solvent to form a suspension;
b. heating the suspension to an elevated temperature to form a solution;
c. adding p-toluenesulfonic acid to the solution obtained in step (b); and
d. isolating anhydrous form of lapatinib ditosylate from the solution of step (c).

19. The process according to claim 18, wherein the ester solvent used in step (a) is selected from ethyl acetate, methyl acetate, isopropyl acetate, tert-butyl methyl acetate, ethyl formate, and mixtures thereof.

20. (canceled)

21. The process according to claim 18, wherein heating in step (b) is at above 25° C.

22. The process according to claim 21, wherein heating in step (b) is at reflux.

23. The lapatinib dioxalate or lapatinib monobesylate salts of claim 1 in the form of a pharmaceutical composition and further comprising a pharmaceutically acceptable carriers, diluents or excipients.

24. (canceled)

25. The composition of claim 23 in the form of a tablet, capsule, suspension, dispersion, or injectable pharmaceutical composition.
